(19) United States
(12) Patent Application Publication
(10) Pub. No.: US 2005/0214889 A1
(43) Pub. Date: Sep. 29, 2005

(54) Apparatus for measuring biodegradability of sample using non-dispersive infrared spectrometry and method of measuring the same

(76) Inventors: Hye-Jung Kang, Gyeonggi-do (KR); Hyang Sun, Gyeonggi-do (KR); Yong-Joo Kim, Gyeonggi-do (KR)

Correspondence Address: SHERIDAN ROSS PC, 1560 BROADWAY, SUITE 1200, DENVER, CO, 80202, US

(21) Appl. No.: 10/528,534

(22) PCT Filed: Nov. 19, 2002
(86) PCT No.: PCT/KR02/02158

§ 371 Date: Jun. 10, 2005

(30) Foreign Application Priority Data
Sep. 23, 2002 (KR) 2002-0057631

(51) Int. Cl. C12Q 1/37; C12M 1/34

(52) U.S. Cl. 435/23; 435/287.1

(57) ABSTRACT

Disclosed is an apparatus for measuring biodegradability of a polymer sample by non-dispersive infrared spectrometry, including a compression pump (2), a first air controlling unit (4), carbon dioxide removing devices (6), a filter (8), a first cooling device (10), at least two composting vessels (12), at least two second cooling devices (10′), at least two second air controlling units (14), at least two non-dispersive infrared gas analyzers 16 and collection units (18), connected in order, and a computer (34) connected to the first air controlling unit, the second air controlling units and the gas analyzers. Using the current apparatus, the amount of carbon dioxide is rapidly, quantitatively and reproducibly measured, and the apparatus is thus used for research and development procedures of biodegradable polymers.

### TECHNICAL FIELD

The present invention pertains to apparatuses and methods for measuring biodegradability of polymers under controlled composting condition. More specifically, the present invention is directed to an apparatus for measuring biodegradability of a polymer and a method of measuring such biodegradability, having the advantages of rapid and quantitative measurement of the amount of carbon dioxide evolved from a composting vessels containing polymer by means of non-dispersive infared (NDIR) spectrometry, and reproducibility of measured results.

### BACKGROUND ART

In general, the term ‘biodegradability’ means the ability of a certain compound to be completely decomposed to simple molecules, for example, CH₄, CO₂ and water, or inorganic salts, by microorganisms and/or under natural environmental conditions. Further, the term ‘biodegradable polymer’ refers to a polymeric compound. which can be decomposed to water, carbon dioxide, methane gas and the like to a decomposition degree of 70% or higher within 45 days by biological functions of microorganisms, not by incinerating but by simply burying the compound used for molded articles, packaging materials, sanitary products, agrochemicals and so on.

Recently, international environmental agreements have been concluded and practiced in various industrial fields for the protection of the global ecosystem. On the other hand, the amount of plastics consumed has increased by 12% every year in Korea, and thus great quantities of plastic wastes have been evolved. Treatment of such wastes has become one of the most serious social problems within the small territory of Korea.

Because of the increasingly stringent environmental standards and the growing environmental awareness of consumers, many producers of all sorts of consumer goods, chemical products etc. are forced to make more environmentally friendly products. In some industrial fields, articles made of biodegradable polymers have been produced.

However, there exist no methods for rapidly, quantitatively and reproducibly determining biodegradability, despite a drastic increase of such biodegradable articles and high commercial availability thereof. Thus, how the biodegradability of various products can be determined becomes of concern.

Biodegradability is typically determined by measuring an amount of products evolved by metabolism of microorganisms which decompose a polymer as an analyte, or an amount of carbon dioxide produced by said microorganisms.

Several methods are known for determining the biodegradability, and one method, the so-called STURM-test, is especially directed towards determining the biodegradability of the products in a water purification device, whereby the production of CO₂ by bacteria is measured in a wet environment. This method using the wet conditions is suitable for less than 1% dried substance at a temperature of 10-20° C., but is unsuitable for determination of biodegradability in a composting device under dry conditions with at least 30-40% dried substance at a temperature of 50° C. or higher.

In WO95/27795, there is disclosed a procedure for measuring the biodegradability of a sample, whereby the sample is placed in a culture solution, the solution is aerated, the quantity of carbon dioxide produced from the solution is determined, and the biodegradability is determined on the basis of the quantity of produced carbon dioxide.

The carbon dioxide, which has been formed is conducted into an alkali solution for precipitation, the electrical conductivity of this solution is measured, and the quantity of carbon dioxide absorbed in the solution is determined on the basis of the electrical conductivity.

In U.S. Pat. No. 5,318,909, there is disclosed a method for the determination of aerobic biodegradability in a composting device of at least one analyte, comprising passing gas which contains oxygen through the composting device including at least two reactors, separately collecting the produced gas from each of said reactors, measuring the amount of carbon dioxide in the produced gas of each of the reactors by means of gas chromatography, and calculating the aerobic biodegradability of at least one analyte based on the measured amount of carbon dioxide in the produced gas of each reactor.

In U.S. Pat. No. 6,143,515, there is disclosed a method for evaluating microbial-degradability of an organic material, by placing the organic material and a microbial source into a reaction column maintained at a fixed temperature under feeding of carbon dioxide-removed saturated water vapor into the column to measure the amount of carbon dioxide formed by the decomposition, placing cellulose together with the same microbial source into another reaction column maintained at the same fixed temperature under feeding of carbon dioxide-removed saturated water vapor into the reaction column to measure the amount of carbon dioxide formed by the decomposition, and comparing the two measured values so as to evaluate the degradability of the organic material.

Additionally, there is provided a titration method for measuring biodegradability of a polymer, by capturing carbon dioxide evolved by the decomposition of the polymer under controlled composting conditions with a mixture solution of potassium hydroxide (KOH) and barium chloride (BaCl₂) filled within a capturing bottle, titrating the KOH solution after the above solution reacts with captured carbon dioxide, to calculate the amount of carbon dioxide evolved, and determining the biodegradability based on such amount of carbon dioxide.

However, as for the above mentioned methods, the measured results depend on the techniques of individual experimenters, therefore resulting in low reproducibility of such results. Furthermore, because experiments are artificially carried out, a time period required for such experiments is long, and quantitative measurements and monitoring in real-time become difficult.

### DISCLOSURE OF INVENTION

Therefore, it is an object of the present invention to overcome the problems encountered in the prior art and to provide a method of measuring biodegradability of a polymer, by measuring concentrations of carbon dioxide formed by metabolism of microorganisms in at least two composting vessels filled with a polymer and/or compost, using non-dispersive infrared gas analyzers.

It is another object of the present invention to provide an apparatus for measuring such biodegradability of a polymer and the advanced testing method that is designed to yield the percentage conversion of carbon in testing material to evolved carbon dioxide as well as the arte of conversion.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a diagram schematically showing a series of processes of measuring biodegradability using non-dispersive infrared spectrometry according to the present invention;

FIG. 2 is a diagram showing a construction of an apparatus for measuring biodegradability using non-dispersive infrared spectrometry according to a primary embodiment of the present invention;

FIG. 3 is a diagram showing a construction of an apparatus for measuring biodegradability using non-dispersive infrared spectrometry according to a secondary embodiment of the present invention;

FIG. 4 is a graph showing biodegradability obtained by means of non-dispersive infrared spectrometry and titration according to the present invention;

FIG. 5 is a graph showing biodegradability obtained by means of non-dispersive infrared spectrometry according to the present invention; and

FIG. 6 is a graph showing an amount of carbon dioxide evolved from compost, measured for a short time period by means of non-dispersive infrared spectrometry.

### BEST MODE FOR CARRYING OUT THE INVENTION

The present invention is characterized by providing an apparatus for measuring biodegradability of a polymer sample, comprising a compression pump 2 for compressing air; a first air controlling unit 4 connected to the compression pump 2 so as to control a flow rate and pressure of compressed air discharged from the compression pump 2; and a plurality of carbon dioxide removing devices 6 connected to the first air controlling unit 4 so as to remove carbon dioxide from the compressed air. The apparatus also comprises a filter 8 connected to the carbon dioxide removing device 6 so as to remove contaminating materials from the carbon dioxide-removed air; a first cooling device 10 connected to the filter 8 so as to cool the air passed through the filter 8; and at least two composting vessels 12 connected to the first cooling device 10 so as to receive the air passed through the first cooling device 10, one of the composting vessels containing the biodegrable polymer sample and compost, and the other composting vessels containing only compost. The apparatus further includes at least two second cooling devices 10′ respectively connected to the composting vessels 12 so as to cool the air discharged from the composting vessels 12; at least two second air controlling units 14 respectively connected to the second cooling devices 10′ so as to control flow rates of the air discharged from the second cooling devices 10′; and at least two non-dispersive infrared gas analyzers 16 respectively connected to the second air controlling units 14 so as to measure concentrations of carbon dioxide in the air discharged from the second air controlling units 14. The apparatus also comprises collection units 18 respectively connected to the gas analyzers 16 so as to collect the air passed through the gas analyzers 16; and a computer 34 connected to the first air controlling unit 4, the second air controlling units 14 and the composting vessels 12 so as to receive data therefrom, and further connected to the gas analyzers 16 so as to interchange data therewith.

In addition, the present invention provides a method of measuring biodegradability of a polymer sample, comprising the following steps of filling the polymer sample and inoculum in one composting vessels of at least two composting vessels maintained at a constant predetermined temperature, and filling only inoculum in the other composting vessels, and compressing external air; removing carbon dioxide from the compressed air; removing contaminating materials from the carbon dioxide-removed air; cooling the contaminating materials-removed air; decomposing the polymer and the compost filled in the composting vessels under an aerobic atmosphere by inflow of the cooled air to each of the composting vessels; cooling air containing carbon dioxide produced from each of the composting vessels; measuring concentrations of carbon dioxide contained in the cooled air by a non-dispersive infrared gas analyzer; transmitting the measured concentration data of carbon dioxide to a computer to calculate biodegradability; and separately collecting carbon dioxide-containing air discharged through each of the gas analyzers.

In order to determine the biodegradability using non-dispersive infrared spectrometry according to the present invention, an amount of carbon dioxide evolved by decomposition of a pure polymer is obtained by subtracting an amount of carbon dioxide produced from a composting vessels containing only the compost from an amount of carbon dioxide evolved by decomposition of the polymer and the compost filled in another composting vessels, followed by calculating the biodegradability of the polymer on the basis of such an amount of carbon dioxide obtained from the decomposition procedure of the polymer.

In such a case, the biodegradability of the polymer sample according to the present invention is calculated as a ratio of amount of carbon dioxide evolved where polymer chains in the polymer sample are broken by inherent biodegradability of the polymer under real buried circumstances, that is to say, composting conditions, versus a maximum theoretically evolved amount of carbon dioxide.

Thus, using the following Equation 1, biodegradability of a sample in each evaluation time period is calculated from an accumulated amount of carbon dioxide discharged, whereby the term “sample” means a biodegradable polymer as an analyte, evaluated in terms of biodegradability.

$${D}_{t} ( \% ) = \frac{{( {CO}_{2} )}_{T} - {( {CO}_{2} )}_{B}}{{ThCO}_{2}} \times 100 \qquad Equation 1$$

wherein,
Dt(%): biodegradability
(CO₂)_{T}: amount of carbon dioxide accumulated from a composting vessels filled with a sample and inoculum
(CO₂)_{B}: averaged amount of carbon dioxide accumulated from another composting vessels filled with only inoculum
ThCO₂: maximum theoretical amount of carbon dioxide evolved from the composting vessels filled with the sample and the inoculum.

Further, the theoretical amount of carbon dioxide above is calculated from the following Equation 2:

$${ThCO}_{2} = {M}_{TOT} \times {C}_{TOT} \times \frac{44}{12} \qquad Equation 2$$

wherein,
M_{TOT}: total solid amount of the sample added to the compost (g)
C_{TOT}: ratio of organic carbon to total solid mass of the sample (g/g)
44: molecular weight of carbon dioxide
12: atomic weight of carbon

In the followers, a description will be given of an apparatus for measuring biodegradability of the polymer and a method of measuring the same, with reference to the attached drawings.

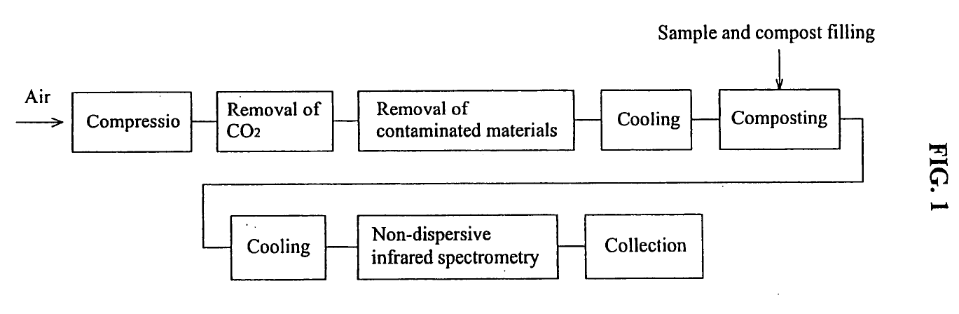

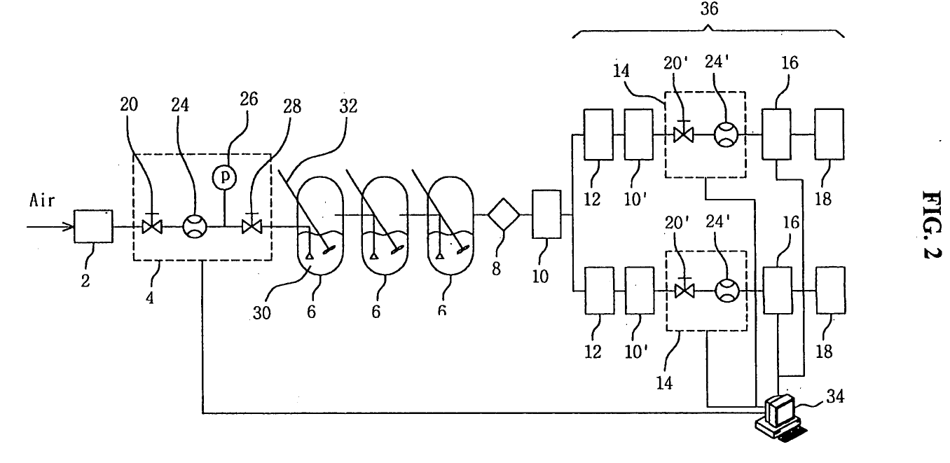

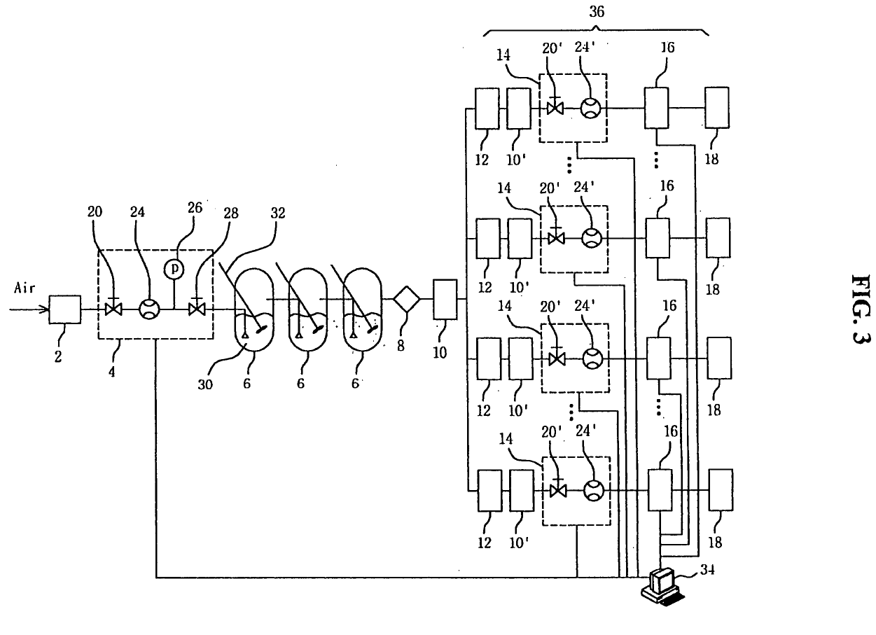

FIG. 1 shows a series of processes for measuring biodegradability using non-dispersive infrared spectrometry according to the present invention, FIG. 2 shows a construction of a biodegradability determination apparatus using non-dispersive infrared spectrometry according to a primary embodiment of the present invention, and FIG. 3 shows a construction of a biodegradability determination apparatus using non-dispersive infrared spectrometry according to a secondary embodiment of the present invention. As presented in FIGS. 1 and 2, the biodegradability of a polymer is determined by measuring the amount of carbon dioxide produced while a polymer is decomposed by microorganisms in a composting vessels, together with the compost.

In the present invention, such amounts of carbon dioxide are measured by means of non-dispersive infrared spectrometry.

In accordance with the primary embodiment of the present invention, the apparatus for use in determination of biodegradability comprises a compression pump 2 for compressing air; a first air controlling unit 4 connected to the compression pump 2 so as to control a flow rate and pressure of the air discharged from the compression pump 2; a plurality of carbon dioxide removing devices 6 connected to the first air controlling unit 4 so as to remove CO₂ from the compressed air; a filter 8 connected to the carbon dioxide removing device 6 so as to remove contaminants from the CO₂-removed air; a first cooling device 10 connected to the filter 8 so as to cool the air passed through the filter 8; at least two composting vessels 12 connected to the first cooling device 10 so as to receive the air passed through the first cooling device 10, one of the composting vessels containing the biodegradable sample and compost, and the other composting vessels containing only compost; at least two second cooling devices 10′ respectively connected to the composting vessels 12 so as to cool the air discharged from the composting vessels 12; at least two second air controlling units 14 respectively connected to the second cooling devices 10′ so as to control flow rates of the air discharged from the second cooling devices 10′; at least two non-dispersive infrared gas analyzers 16 respectively connected to the second air controlling units 14 so as to measure concentrations of CO₂ in the air discharged from the second air controlling units 14; collection units 18 for collecting the air passed through the gas analyzers 16; and a computer connected to the first air controlling unit 4, the second air controlling units 14, a computer 34 connected to the composting vessels 12 so as to receive data therefrom, and further connected to the gas analyzers 16 so as to interchange data.

As such, a sequentially connected composting vessels 12, second cooling device 10′, second air controlling unit 14, non-dispersive infrared gas analyzer 16 and collection unit 18 forms a sampling part 36, and two or more sampling parts are installed in an apparatus for measuring the biodegradability.

In accordance with an embodiment of the present invention, there is provided an apparatus equipped with multiple sampling parts 36, in which the air discharged from the first cooling device 10 is fed to each of the used sampling parts 36 (FIG. 3). Further, the second air controlling unit 14 and the non-dispersive infrared gas analyzer 16 included in each of the sampling parts 36 are connected to the computer 34.

The first and the second air controlling units 4 and 14 are responsible for providing the air at a constant predetermined flow rate and pressure, so as to accurately measure the amount of carbon dioxide by means of an optical sensor of the gas analyzer 16. Thus, the first air controlling unit 4 is composed of a needle valve 20, a flow meter 24, a manometer 26 and a check valve 28, with the aim of controlling the flow rate and pressure of air flowing into the carbon dioxide removing device 6, while the second air controlling unit 14 includes a needle valve 20′ and a flow meter 24′ in order to control the flow rate of air flowing into the gas analyzer 16.

As the flow meters 24 and 24′ in the first and the second air controlling units 4 and 14, a mass flow controller is preferably used. With the intention of compensation of pressure loss at the junction of each of the carbon dioxide removing device 6 and the composting vessels 12, such a flow controller is equipped with an equal percentage valve operated by a proportional-integral-derivative (PD) control manner, and thus maintains a flow rate and pressure required for operation thereof at a constant predetermined level, in which the equal percentage valve is connected to the computer 34 and is under the control of the computer.

As for the carbon dioxide removing device 6, any device may be used as long as carbon dioxide may be continuously removed from external air flowing into the apparatus. Preferably, a vessel packed with sodium hydroxide solution 30 is used. As necessary, multiple vessels packed with sodium hydroxide solution 30 may be connected and used, in which a stirrer 32 is provided to increase contact between inflowing air and sodium hydroxide solution 30.

A carbon filter, a hepa filter, a depth filter, a membrane filter, etc exemplify the filter 8 for removal of contaminating materials such as harmful gases contained in air. Among them, a carbon filter is preferred.

The cooling devices 10 and 10′ act to condense moisture present in air to water by cooling the air. The first cooling device 10 is connected at the rear of the filter 8 to remove moisture in air flowing into the composting vessels 12, while the second cooling device 10′ functions to cool moisture in air discharged from the composting vessels 12 and to recover cooled moisture into a water receptacle positioned at a bottom portion in the composting vessels 12, thereby feeding only dehumidified air to a measuring unit. The cooling devices 10 and 10′ are maintained at 0-10° C., and preferably, at 1-8° C.

The composting vessels 12 is filled with the polymer sample and/or the inoculum, and has a water receptacle at its bottom portion. Water is charged in such a water receptacle and aerated, whereby moisture content of the sample and/or the seeded compost filled in the composting vessels 12 is constantly maintained at a predetermined level.

Each composting vessels 12 is maintained at a temperature suitable for metabolism of microorganisms under an aerobic condition, for instance, 55-60° C. As such, the sample is filled in the amount of about 5% by dry weight of the compost. Cattle feedstuff, sawdust or combinations thereof exemplify the compost filled in composting vessels 12. Moisture content in such compost ranges from 50 to 80%, and preferably, from 60 to 70%, and more preferably, 65%. In addition, microorganisms for decomposing the polymer sample are seeded to the compost exemplified above and placed into the composting vessels 12 together with the above compost.

As mentioned above, before the compost is introduced into the composting vessels 12, the following seeding process is performed: microorganisms are seeded to a certain compost and sufficiently grown in an incubator; then, when the growth curve of the microorganisms reaches steady state, a small quantity of compost containing such microorganisms is separated from the incubator and then added to the pure compost. Thereby, the inoculum to be filled in the composting vessels 12 is prepared.

In the present invention, the compost that contains microorganisms grown in the incubator, which is added in a predetermined amount to compost before being placed into the composting vessels 12, is referred to as seeding compost.

A standard amount of carbon dioxide required for calculation of biodegradability is based on the amount of carbon dioxide evolved from the composting vessels 12 filled with only the compost.

The non-dispersive infrared gas analyzer 16 functions to measure a concentration of carbon dioxide produced by a biological decomposition procedure in the composting vessels 12. Any device may be used as long as the concentration of carbon dioxide can be measured.

With the aim of continuously measuring the concentration of carbon dioxide in air discharged from the second air controlling unit 14 located at the front of the gas analyzer 16, the gas analyzer 16 is provided with an optical sensor including a light-emitting part and a light-receiving part positioned in a flow passage of air. When light is irradiated from the light-emitting part in the optical sensor, a part thereof is absorbed at a predetermined wavelength. The measured results are transmitted to the computer 34, whereby the amount of carbon dioxide in air is quantitatively measured in real time.

As such, the light-emitting part serves to irradiate light to a desired subject and is exemplified by a light emitting diode, while the light-receiving part functions to absorb the irradiated light, and a photo diode may be used for such absorption.

The computer 34, which is interconnected to the gas analyzer 16, functions to analyze and record the measured data input from the gas analyzer 16. As well, the computer 34 is further connected to the first air controlling unit 4, the second air controlling unit 14 and the composting vessels 12, and controls flow rates, pressures, temperatures and water contents of the above mentioned units in real-time, and monitors them under a remote control using TCP/IP communication.

An operating principle of the apparatus of the present invention for use in determination of the biodegradability by non-dispersive infrared spectrometry is described below.

About 3.5 g of a polymer sample is charged into a first composting vessels 12, together with 200 g of compost seeded with microorganisms for decomposing the polymer sample. The compost is obtained by adding a small amount of the seeding compost to cattle feedstuff, sawdust or combinations thereof. Only the compost as defined above is filled in a second composting vessels 12. Thereafter, external air flows into the compression pump and is compressed. Such compressed air is fed to the first air controlling unit 4. As such, by means of the needle valve 20, the flow meter 24, the manometer 26 and the check valve 28 included in the first air controlling unit 4, the compressed air flows at constant flow rate.

Thereafter, the air flowing through the first air controlling unit 4 is passed through the carbon dioxide removing device 6 which is connected at the back of the first air controlling unit 4, thereby removing carbon dioxide. Such carbon dioxide-removed air streams into the filter 8 to remove contaminating materials from the air. Such air flows into the first cooling device 10 and is cooled to 0-10° C. When air flowing into the first cooling device 10 is cooled, water is condensed therefrom, which is then recovered to water receptacles provided at the bottom portions of the composting vessels 12 which are connected at the back of the first cooling device 10, or is discharged outside of the first cooling device 10.

The cooled air flows into the first composting vessels 12 containing the sample and the compost and the second composting vessels 12 containing only the compost. Each composting vessels 12 is maintained at 55-65° C., and water filled in the water receptacle of the bottom portion in each composting vessels 12 is aerated, whereby water is provided to the sample and/or the inoculum in the composting vessels. Microorganisms seeded to the compost, to produce carbon dioxide, decompose the sample and/or the compost in the baths 12.

Air containing carbon dioxide discharged from each composting vessels 12 flows into the second cooling device 10′ which is connected at the rear of each composting vessels 12, and then cooled. Thereby, moisture in air flowing to the second cooling device 10′ is recovered as water.

Such water is fed to the water receptacles at the bottom portions in the composting vessels 12 which are connected at the front of the second cooling devices 10′, or is discharged outside of the second cooling devices 10′.

Air containing carbon dioxide discharged from each of the second cooling devices 10′ streams into the second air controlling units 14 which are connected to the second cooling devices 10′. Such air is passed through the predetermined flow passage equipped with the optical sensor of the gas analyzer 16 connected to each of the second air controlling units 14 while being controlled by the needle valve 20′ and the flow meter 24′ constituting each second air controlling unit 14, thus collecting the discharged air in the collection unit 18.

The optical sensors of the gas analyzers 16 provided at the predetermined passage measure the concentrations of carbon dioxide in air passed through the second air controlling units 14 and the measured data to the computer 34 interconnected with the gas analyzers 16. The concentration data are transmitted computer 34 is calculated by the computer 34 to determine the biodegradability. The biodegradability is calculated from the above Equations 1 and 2, based on the measured amount of carbon dioxide.

The optical sensor of each gas analyzer 16 should be periodically corrected on the basis of a calibration curve, to obtain accurate experimental results. Therefore, two standard concentration gases having different concentrations, for example, 1000, 4000, 10000 or 50000 ppm, are prepared, and output signals for different gas concentrations are measured and compared, and errors are corrected, determining such a calibration curve. The above standard concentration gas means carbon dioxide.

Further, when the non-dispersive infrared gas analyzer 16 do not accurately estimate errors varying with time, a specialized working standard substance having a known concentration is passed through the gas analyzer for a shorter evaluation time interval than the standard substance, thus correcting such errors.

In the first air controlling unit 4, the second air controlling units 14 and the composting vessels 12 that are connected to the computer 34, pressures and flow rates of air, and temperatures of the composting vessels 12 are recorded to the computer 34, and suitable operating conditions are concurrently controlled by the computer 34.

To improve reproducibility of results according to determination of the biodegradability, multiple sampling parts 36 are installed in the present apparatus, each of which includes a sequentially connected composting vessels 12, second cooling device 10′, second air controlling unit 14, non-dispersive infrared gas analyzer 16 and collection unit 18. In the multiple sampling parts 36, the analyte under the same conditions is introduced into each of the composting vessels 12, and the amount of carbon dioxide discharged from each of the baths 12 is measured and these values are averaged, from which the biodegradability is determined. Meanwhile, various samples are placed together with the compost in the composting vessels in multiple sampling parts 36, and thus the biodegradability of each sample can be measured by a single experiment.

A better understanding of the present invention may be obtained in light of the following examples which are set forth to illustrate, but are not to be construed to limit the present invention.

### EXAMPLE 1

A compression pump [coolant pump, YUILAIR INSTRUMENT, Korea], a needle valve [air controller, SANYANG AUTO INSTURMENT, Korea], a flow meter [mass flow controller, KOFLOC, Japan], a manometer [pressure gauge, SHINYANG AUTO INSTURMENT, Korea] and a check valve were connected in series as shown in FIG. 2, after which 3 vessels containing 10N NaOH aqueous solution and equipped with a stirrer were connected to one another at the rear of the check valve.

Thereafter, a filter [chemical deodorization filter, Joul Heater Co. Ltd., Korea] was connected at the back of the terminal vessel filled with 10N NaOH aqueous solution, and a cooling device [bath circulator, JEIO TECH, Korea] was mounted to the back of the filter [chemical deodorization filter, Joule Heater Co. Ltd., Korea].

Two 50 L composting vessels were connected to the cooling device [bath circulator, JEIO TECH, Korea], one of which was filled with 200 g of seeding compost-containing compost and 3.5 g of cellulose [SIGMACELL type 20, SIGMA, USA], the other of which contained only 200 g of compost. Each composting vessels was maintained at 55° C. The compost having water content of 65%, pH 8.61, was seeded with microorganisms for decomposing a polymer before being filled in the composting vessels.

Air was circulated from the cooling device [bath circulator, JEIO TECH, Korea] to each composting vessels at 45 sccm.

The needle valve [air controller, SHINYANG AUTO INSTURMENT , Korea] and the flow meter [mass flow controller, KOFLOC, Japan] were connected at the rear of each of the composting vessels, and optical sensors of non-dispersive infrared gas analyzers [A-SENSE-D, SenseAir, Sweden] were mounted to flow passages of the air through the flow meters [mass flow controller, KOFLC, Japan]. Further, a collection unit for collecting the air passed through each optical sensor was located at a terminal end of each flow passage of air.

Then, the gas analyzers [A-SENSE-D, SenseAir, Sweden] were interconnected to a computer to interchange data. With the intention of controlling air flow and temperature of the composting vessels, the flow meters [mass flow controller, KOFLOC, Japan] and the composting vessels sensors were connected to the computer.

Using the gas analyzers [A-SENSE-D, SenseAir, Sweden], amounts of carbon dioxide evolved from the composting vessels were measured to determine biodegradability of the polymer sample.

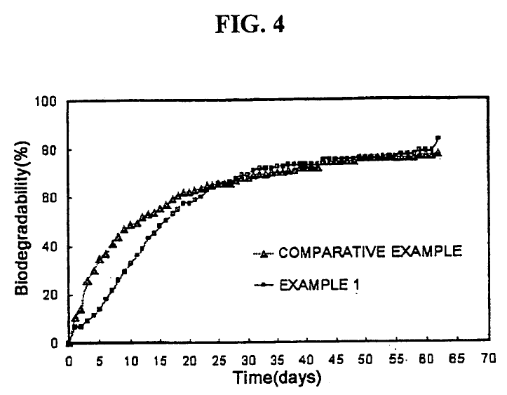

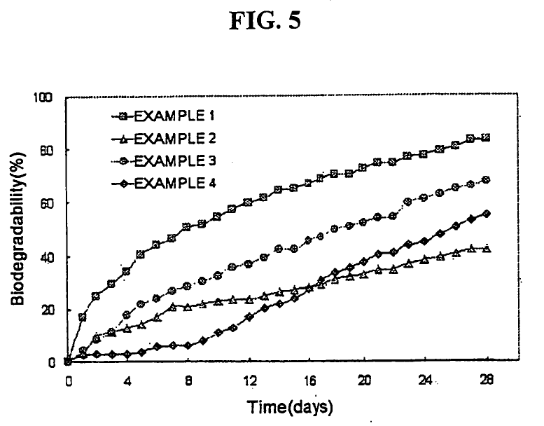

The results are shown in Table 1, and in the attached FIGS. 4 and 5.

### EXAMPLE 2

The present example was carried out in the same manner as in the above Example 1, except that PBAT DSE [IRe Chemical Ltd., Korea] was used instead of cellulose [SIGMACELL type 20, SIGMA, USA].

The results are shown in Table 1, and in the attached FIG. 5.

### EXAMPLE 3

The present example was carried out in the same manner as in the above Example 1, except that PBAT MSA [IRe Chemical Ltd., Korea] was used instead of cellulose [SIGMACELL type 20, SIGMA, USA].

The results are shown in Table 1 and in the attached FIG. 5.

### EXAMPLE 4

The present example was carried out in the same manner as in the above Example except that PBAT NC [IRe Chemical Ltd., Korea] was used instead of cellulose [SIGMACELL type 20, SIGMA, USA].

The results are shown in Table 1, and in the attached FIG. 5.

TABLE I

| | | Theoretical CO₂ |
|---|---|---|
| Ex. No. | Carbon amount (%) | generation amount (mg) |
| 1 | 41.5318 | 5329.8 |
| 2 | 61.5235 | 7894.95 |
| 3 | 61.1175 | 7843.85 |
| 4 | 62.2201 | 7984.9 |

### COMPARATIVE EXAMPLE

Biodegradability Determination using Titration

A compression pump [coolant pump, YUILAIR INSTURMENT, Korea], a needle valve [air controller, SANYANG AUTO INSTURMENT, Korea], a flow meter [mass flow controller, KOFLOC, Japan], a manometer [pressure gauge, SHINYANG AUTO INSTURMENT, Korea] and a check valve were connected in series, after which 3 vessels containing 10N NaOH aqueous solution and equipped with stirrers were connected to one another at the rear of the check valve.

Then, a filter [chemical deodorization filter, Joul Heater Co. Ltd., Korea] was connected to the terminal vessel containing 10 N NaOH aqueous solution, and a cooling device [bath circulator, JEIO TECH, Korea] was mounted to the back of the filter [chemical deodorization filter, Joule Heater Co. Ltd., Korea].

Two 50 L composting vessels were connected to the cooling device [bath circulator, JEIO TECH, Korea], one of which was filled with 200 g of the compost under the same condition as in the above Example 1, and 3.5 g of cellulose [SIGMACELL type 20, SIGMA, USA], the other of which contained only 200 g of the above compost. Each composting vessels was maintained at 55° C. The compost was seeded with microorganisms for decomposing the polymer before being filled into the baths.

Thereafter, a collection unit connected to each of the baths collected carbon dioxide-containing air discharged from each of the composting vessels. As such, the collection unit was previously filled with a mixture of 130 ml of 0.4N KOH and 26 ml of 2N BaCl₂ aqueous solution.

A stirrer was equipped in the collection unit to completely react carbon dioxide in air with said aqueous solution in the unit. After completion of the reaction, 12 ml of the reacted aqueous solution was introduced into a 100 ml Erlenmeyer flask.

With stirring the Erlenmeyer flask 38, 2-3 drops of phenolphthalein aqueous solution was added dropwise into the flask and the solution in the flask was titrated with 0.2N aqueous hydrochloric acid solution until the solution in the flask was colorless. Carbon dioxide collected in the collection unit reacts as represented by the following Reaction 1. Practically, 12 ml of the reacted aqueous solution includes 10 ml of aqueous potassium hydroxide solution. In the case where no carbon dioxide is collected, a titer of 0.2 N HCl aqueous solution used for titration of 10 ml of KOH solution is 20 ml. And, the amount of aqueous potassium hydroxide which reacts with a titrating solution is the unreacted KOH remaining after practically reacting with carbon dioxide. Through titration, the amount of the unreacted KOH aqueous solution can be determined. Thus, the amount of carbon dioxide can be obtained by subtracting a titer of HCl aqueous solution used for titration from 20 ml.

Reaction 1
2KOH+CO₂→2K+CO₃²⁻+H₂O
Ba(Cl)₂+CO₃²⁻→BaCO₃+2Cl⁻
wherein KOH:CO₂ reacts at a ratio of 2:1.

The used amount of the aqueous solution in the flask corresponds to 1/13 of the original aqueous solution in the collection unit. Hence, the practical amount of carbon dioxide was obtained by multiplying 13 to the above obtained value.

Based on the measured carbon dioxide amount, the biodegradability was calculated from the above Equations 1 and 2.

The results are shown in the attached FIG. 4.

Experiment

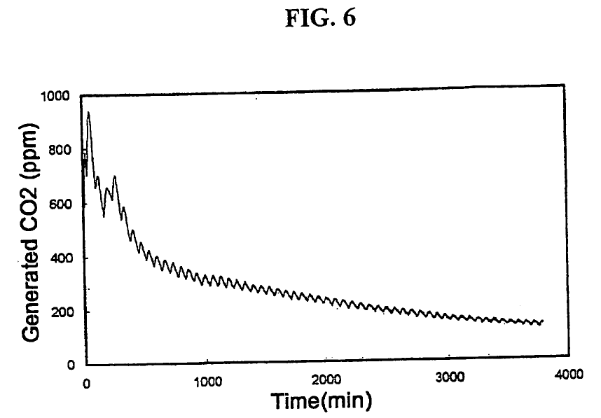

Referring to FIG. 6, there is shown an experimental result of the amount of carbon dioxide evolved from the compost, measured for a short term period by non-dispersive infrared spectrometry according to the above Example 1.

As seen in FIG. 6, an initial amount of carbon dioxide close to 1000 ppm was decreased to 200 ppm after only 3 days. This means that over 80% of cellulose [SIGMACELL type 20, SIGMA, USA] used in the above Example 1 was biodegraded within 3 days, and can be referred to as a biodegradable polymer.

Whether the sample is a biodegradable polymer or not can be determined by means of the biodegradability determination apparatus of the present invention, which is advantageous in terms of shorter testing periods, compared to conventional apparatuses requiring 1 week or longer.

### INDUSTRIAL APPLICABILITY

According to the biodegradability measuring method of the present invention, the amount of carbon dioxide is rapidly, quantitatively and reproducibly measured, using non-dispersive infrared spectrometry, and thus the method is properly used for research and development procedures of biodegradable polymers.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. An apparatus for measuring biodegradability of polymer sample, comprising:
a compression pump for compressing air;
a first air controlling unit connected to the compression pump so as to control a flow rate and pressure of compressed air discharged from the compression pump;
a carbon dioxide removing device connected to the first air controlling unit so as to remove carbon dioxide from the compressed air;
a filter connected to the carbon dioxide removing device so as to remove contaminating materials from the carbon dioxide-removed air,
a first cooling device connected to the filter so as to cool the air passed through the filter;
at least two composting vessels connected to the first cooling device so as to receive the air passed through the first cooling device, one of the composting vessels containing the biodegrable polymer sample and compost, and the other composting vessels containing only compost;
at least two second cooling devices respectively connected to the composting vessels so as to cool the air discharged from the composting vessels;
at least two second air controlling units respectively connected to the second cooling devices so as to control flow rates of the air discharged from the second cooling devices;
at least two non-dispersive infrared gas analyzers respectively connected to the second air controlling units so as to measure concentrations of carbon dioxide in the air discharged from the second air controlling units;
collection units respectively connected to the gas analyzers so as to collect the air passed through the gas analyzers; and
a computer connected to the first air controlling unit, the second air controlling units and the composting vessels so as to receive data therefrom and further connected to the gas analyzers so as to interchange data therewith.

2. The apparatus as set forth in claim 1, wherein the first air controlling unit comprises a needle valve, a flow meter, a manometer, and a check valve.

3. The apparatus as set forth in claim 1, wherein each of the second air controlling units comprises a needle valve and a flow meter.

4. The apparatus as set forth in claim 1, wherein the carbon dioxide removing device comprises a vessel containing a sodium hydroxide solution and equipped with a stirrer.

5. A method of measuring biodegradability of a polymer sample, comprising the steps of:
filling the polymer sample and inoculum in one composting vessels of at least two composting vessels maintained at a. constant predetermined temperature, and filling only inoculum in the other composting vessels, and compressing external air;
removing carbon dioxide in the compressed air of the above step (i);
removing contaminating materials from the carbon dioxide-removed air of the above step (ii);
cooling the contaminating materials-removed air of the above step (iii);
decomposing the polymer and the compost filled in the composting vessels by microorganisms under an aerobic atmosphere by inflow of the cooled air of the above step (iv) to each of the composting vessels;
cooling air containing carbon dioxide produced from each of the composting vessels of the above step (v);
measuring a concentration of carbon dioxide contained in each of the cooled air of the above step (vi), by non-dispersive infrared spectrometry;
transmitting the concentration data of carbon dioxide measured in the above step (vii) to a computer, to calculate biodegradability; and
separately collecting carbon dioxide-containing air discharged after the above step (viii).
